(19) United States
(12) Patent Application Publication
(10) Pub. No.: US 2024/0050764 A1
(43) Pub. Date: Feb. 15, 2024

(54) THERAPEUTIC EYEGLASSES

(71) Applicant: Keith COLLEY, Kincardine (CA)

(72) Inventor: Keith COLLEY, Kincardine (CA)

(21) Appl. No.: 18/356,614

(22) Filed: Jul. 21, 2023

Related U.S. Application Data
(63) Continuation of application No. PCT/CA2022/050369, filed on Mar. 11, 2022.
(60) Provisional application No. 63/160,362, filed on Mar. 12, 2021.

(51) Int. Cl. A61N 5/06

(52) U.S. Cl. CPC A61N 5/06; A61N 2005/0648

(57) ABSTRACT

Therapeutic eyeglasses are disclosed. In one embodiment, therapeutic eyeglasses comprises: a frame comprising a frame front; a pair of temples attached to respective ends of the frame front; and a pair of lenses received in the frame front, wherein each lens comprises a light modifying area and a circular light transmitting area within the light modifying area, the circular light transmitting area proximate to a centre of the lens, wherein the diameter of the circular light transmitting area is between 10 and 25 mm, and a linear distance between a centre of the circular light transmitting area of the lenses is between 40 mm and 80 mm.

### RELATED APPLICATION DATA

The present application is a continuation of international PCT patent application no. PCT/CA2022/050369 filed Mar. 11, 2022, which claims priority to, and the benefit of, provisional U.S. patent application No. 63/160,362, filed Mar. 12, 2021, the content of both of these documents being incorporated herein by reference.

### TECHNICAL FIELD

The present disclosure relates to therapeutic eyeglasses.

### BACKGROUND

Vision-related conditions, such as visual impairment and mental or psychological problems associated with vision, are common problems that affect many people. Vision-related conditions include light sensitivity, eyestrain, stress, anxiety, panic attacks, concentration, and attention deficit hyperactivity disorder (ADHD). Therapeutic eyeglasses which treat various vision-related conditions are desired.

### BRIEF DESCRIPTION OF THE DRAWINGS

FIGS. 1 to 7 illustrate therapeutic eyeglasses in accordance with a first embodiment of the present disclosure. FIG. 1 is a front perspective view of the first embodiment of the therapeutic eyeglasses. FIG. 2 is a rear perspective view of the therapeutic eyeglasses of FIG. 1. FIG. 3 is a right side view of the therapeutic eyeglasses of FIG. 1. FIG. 4 is a front view of the therapeutic eyeglasses of FIG. 1. FIG. 5 is a left side view of the therapeutic eyeglasses of FIG. 1. FIG. 6 is a front view of the therapeutic eyeglasses of FIG. 1. FIG. 7 is a top view of the therapeutic eyeglasses of FIG. 1.

FIGS. 8 to 14 illustrate therapeutic eyeglasses in accordance with a second embodiment of the present disclosure. FIG. 8 is a front perspective view of the second embodiment of the therapeutic eyeglasses. FIG. 9 is a rear perspective view of the therapeutic eyeglasses of FIG. 8. FIG. 10 is a right side view of the therapeutic eyeglasses of FIG. 8. FIG. 11 is a front view of the therapeutic eyeglasses of FIG. 8. FIG. 12 is a left side view of the therapeutic eyeglasses of FIG. 8. FIG. 13 is a front view of the therapeutic eyeglasses of FIG. 8. FIG. 14 is a top view of the therapeutic eyeglasses of FIG. 8.

FIGS. 15 to 21 illustrate therapeutic eyeglasses in accordance with a third embodiment of the present disclosure. FIG. 15 is a front perspective view of the third embodiment of the therapeutic eyeglasses. FIG. 16 is a rear perspective view of the therapeutic eyeglasses of FIG. 15. FIG. 17 is a right side view of the therapeutic eyeglasses of FIG. 15. FIG. 18 is a front view of the therapeutic eyeglasses of FIG. 15. FIG. 19 is a left side view of the therapeutic eyeglasses of FIG. 15. FIG. 20 is a front view of the therapeutic eyeglasses of FIG. 15. FIG. 21 is a top view of the therapeutic eyeglasses of FIG. 15.

FIGS. 22 to 28 illustrate therapeutic eyeglasses in accordance with a fourth embodiment of the present disclosure. FIG. 22 is a front perspective view of the fourth embodiment of the present disclosure. FIG. 23 is a rear perspective view of the therapeutic eyeglasses of FIG. 22. FIG. 24 is a right side view of the therapeutic eyeglasses of FIG. 22. FIG. 25 is a front view of the therapeutic eyeglasses of FIG. 22. FIG. 26 is a left side view of the therapeutic eyeglasses of FIG. 22. FIG. 27 is a front view of the therapeutic eyeglasses of FIG. 22. FIG. 28 is a top view of the therapeutic eyeglasses of FIG. 15.

FIGS. 29 to 35 illustrate therapeutic eyeglasses in accordance with a fifth embodiment of the present disclosure FIG. 29 is a front perspective view of the fifth embodiment of the present disclosure. FIG. 30 is a rear perspective view of the therapeutic eyeglasses of FIG. 29. FIG. 31 is a right side view of the therapeutic eyeglasses of FIG. 29. FIG. 32 is a front view of the therapeutic eyeglasses of FIG. 29. FIG. 33 is a left side view of the therapeutic eyeglasses of FIG. 29. FIG. 34 is a front view of the therapeutic eyeglasses of FIG. 29. FIG. 35 is a top view of the therapeutic eyeglasses of FIG. 29.

### DETAILED DESCRIPTION OF EXAMPLE EMBODIMENTS

The present disclosure is made with reference to the accompanying drawings, in which embodiments are shown. However, many different embodiments may be used, and thus the description should not be construed as limited to the embodiments set forth herein. Wherever possible, the same reference numbers are used in the drawings and the following description to refer to the same elements. Lastly, elements referred to in the singular may be plural and vice versa, except where indicated otherwise either explicitly or inherently by context.

The present disclosure provides therapeutic eyeglasses which reduce the field of the view of the wearer. The therapeutic eyeglasses comprise lenses that are light modifying except for a light transmitting area (also referred to as a window) within the light modifying area. The light modifying area may be blocking, occluding, obscuring or tinted whereas the light transmitting area is correspondingly non-light blocking, non-occluding, non-obscuring or non-tinted. The light modifying area may be formed by a durable coating on the lens, a film adhered to the lens, a tinting of the lens, an abrading of the lens, or otherwise formed as a fixed part of the lens. The light transmitting area may be circular or elongate. When circular, the diameter of the circular light transmitting area is between 10 and 25 mm and the linear distance between a centre of the circular light transmitting area is between 40 mm and 80 mm. The therapeutic eyeglasses may have a curved or wraparound frame and lenses. The therapeutic eyeglasses may have top and/or side guards/shields to block peripheral light.

In accordance with a first aspect of the present application, there is provided therapeutic eyeglasses, comprising: a frame comprising: a frame front; and a pair of temples attached to respective ends of the frame front; and a pair of lenses received in the frame front, wherein each lens comprises a light blocking area and a circular light transmitting area within the light blocking area, the circular light transmitting area proximate to a centre of the lens, wherein the diameter of the circular light transmitting area is between 10 and 25 mm, and a linear distance between a centre of the circular light transmitting area of the lenses is between 40 mm and 80 mm.

In accordance with a second aspect of the present application, there is provided therapeutic eyeglasses, comprising: a frame comprising: a frame front; and a pair of temples attached to respective ends of the frame front; and a pair of lenses received in the frame front, wherein each lens comprises a tinted area and a circular non-tinted area within the tinted area, the non-tinted area proximate to a centre of the lens, wherein the diameter of the circular non-tinted area is between 10 and 25 mm, and a linear distance between a centre of the non-tinted area of the lenses is between 40 mm and 80 mm.

In accordance with a third aspect of the present application, there is provided therapeutic eyeglasses, comprising: a frame comprising: a frame front; and a pair of temples attached to respective ends of the frame front; and a pair of lenses received in the frame front, wherein each lens comprises an obscuration area and a circular light transmitting area within the obscuration area, the circular light transmitting area proximate to a centre of the lens, wherein the diameter of the circular light transmitting area is between 10 and 25 mm, and a linear between a centre of the circular light transmitting area of the lenses is between 40 mm and 80 mm.

In accordance with a fourth aspect of the present application, there is provided therapeutic eyeglasses, comprising: a frame comprising: a frame front; and a pair of temples attached to respective ends of the frame front; and a pair of lenses received in the frame front, wherein each lens comprises a light modifying area and a circular light transmitting area within the light modifying area, the circular light transmitting area proximate to a centre of the lens, wherein the diameter of the circular light transmitting area is between 10 and 25 mm, and a linear distance between a centre of the circular light transmitting area of the lenses is between 40 mm and 80 mm.

In some examples of the fourth aspect, the diameter of the circular light transmitting area is between 15 and 20 mm, and some examples 15 mm, and in some examples 20 mm.

In some examples of the fourth aspect, the distance between the centres of the circular light transmitting area is between 55 mm and 65 mm and some examples between 57.5 mm and 62.5 mm.

In some examples of the fourth aspect, the diameter of the circular light transmitting area is 16 mm, wherein the distance between the centres of the circular light transmitting area is 58 mm.

In some examples of the fourth aspect, the diameter of the circular light transmitting area is 20 mm, wherein the distance between the centres of the circular light transmitting area is 61 mm.

In some examples of the fourth aspect, the circular light transmitting area 144 is located 2 mm to 15 mm below the centre of the respective lens 40 in some examples, 2 mm to 10 mm in some examples, 2 mm to 7.5 mm in some examples, and 2 mm to 5 mm in some examples.

In accordance with a fifth aspect of the present application, there is provided therapeutic eyeglasses, comprising: a frame comprising: a frame front comprising a bridge; and a pair of temples attached to respective ends of the frame front; and a pair of lenses received in the frame front, wherein each lens comprises a light modifying area and an elongate light transmitting area within the light modifying area, the elongate light transmitting area comprising a curved portion having a centre and an elongate portion extending laterally from the curved portion to an inner edge of the lens proximate to the bridge, the centre of the curved portion of the elongate light transmitting area proximate to a centre of each lens, wherein a linear distance between the centre of the curved portion of the elongate light transmitting area of the lenses is between 40 mm and 80 mm.

In some examples of the fifth aspect, the curved portion is a semi-circular portion.

In some examples of the fifth aspect, the diameter of the semi-circular portion is between 10 and 25 mm, and in some examples, between 15 and 20 mm.

In some examples of the fifth aspect, the elongate light transmitting area is located 2 mm to 15 mm below the centre of the respective lens 40 in some examples, 2 mm to 10 mm in some examples, 2 mm to 7.5 mm in some examples, and 2 mm to 5 mm in some examples.

In some examples of the fourth and fifth aspect, the light modifying area of each lens is formed by a durable coating on the lens.

In some examples of the fourth and fifth aspect, the light modifying area of each lens is formed by a film adhered on the lens.

In some examples of the fourth and fifth aspect, the light modifying area is a light blocking area that prevents substantially all light from passing through the lens.

In some examples of the fourth and fifth aspect, the light modifying area is a tinted area and the circular light transmitting area is a circular non-tinted area within the tinted area.

In some examples of the fourth and fifth aspect, the light modifying area is an obscuration area.

In some examples of the fourth and fifth aspect, the obscuration area of each lens is provided by an abraded surface.

In some examples of the fourth and fifth aspect, the obscuration area obscures the vision of a wear.

In some examples of the fourth and fifth aspect, the obscuration area scatters and/or diffuses light such that substantially all objects viewed through the obscuration area are undiscernible by the wearer.

In some examples of the fourth and fifth aspect, the obscuration area has a haze of 25% or more, in some examples 35% or more, and more in some examples 50% or more.

In some or all examples, the frame and lenses are curved.

In some or all examples, the therapeutic eyeglasses are wraparound eyeglasses.

In some or all examples, the frame further comprises a light blocking top shield extending from a top of the frame front to block peripheral light from the eyes of a wearer.

In some or all examples, the frame further comprises light blocking side shields extending from each of the temples to block peripheral light from the eyes of a wearer.

In some examples, the obscuration area of each lens is provided by an abraded surface.

In some examples, the obscuration area obscures the vision of a wear.

In some examples, the obscuration area scatters and/or diffuses light such that substantially all objects viewed through the obscuration area are undiscernible by the wearer.

In some examples, the obscuration area has a haze of 25% or more, in some examples 35% or more, and more in some examples 50% or more.

In accordance with a further aspect of the present application, there is provided use of the therapeutic eyeglasses of the above-described aspects for the treatment of increase mental focus and concentration.

In accordance with a further aspect of the present application, there is provided use of the therapeutic eyeglasses of the above-described aspects for the treatment of treat stress, anxiety, and panic attacks.

In accordance with a further aspect of the present application, there is provided use of the therapeutic eyeglasses of the above-described aspects for the treatment of attention deficit hyperactivity disorder (ADHD).

In accordance with a further aspect of the present application, there is provided use of the therapeutic eyeglasses of the above-described aspects for reducing eye strain.

In accordance with a further aspect of the present application, there is provided use of the therapeutic eyeglasses of the above-described aspects for the treatment of light sensitivity.

In accordance with a further aspect of the present application, there is provided use of the therapeutic eyeglasses of the above-described aspects for sports training.

In accordance with further aspects of the present application, there is provided methods of making therapeutic eyeglasses as described herein. In one example, there is provided a method of making therapeutic eyeglasses, comprising: applying a mask to a surface of each lens in a pair of lenses for therapeutic eyeglasses, wherein each mask has a size and shape corresponding to a size and shape of a light transmitting area of a respective lens; abrading the surface of each lens; removing the mask from each lens in the pair of lenses; and inserting the pair of lenses into a frame of the therapeutic eyeglasses. In one example, there is provided a method of making therapeutic eyeglasses, comprising: inserting a pair of lenses into a frame of the therapeutic eyeglasses; applying a mask to a surface of each lens in the pair of lenses of the therapeutic eyeglasses, wherein each mask has a size and shape corresponding to a size and shape of a light transmitting area of a respective lens; abrading the surface of each lens; and removing the mask from each lens in the pair of lenses.

In some or all examples, the masks are circular.

In some or all examples, the masks are elongate.

In some or all examples, the elongate masks comprise a curved portion and an elongate portion.

In some or all examples, the curved portion is a semi-circular portion.

In some or all examples, the diameter of the semi-circular portion is between 10 and 25 mm, and in some examples, between 15 and 20 mm, and some examples 15 mm, and in some examples 20 mm.

In some or all examples, a linear distance between the centre of the curved portion of the elongate light transmitting area of the lenses is between 40 mm and 80 mm, and in some examples between 55 mm and 65 mm, and in some examples between 57.5 mm and 62.5 mm.

In some or all examples, the abrading comprises abrasive blasting.

In some or all examples, the lenses are subjected to abrasive blasting until a threshold level of abrasion is achieved.

In some or all examples, the threshold level of abrasion is achieved when a threshold level of obscuration is achieved.

In some or all examples, the threshold level of obscuration is achieved when light passing through the obscuration area is sufficiently scattered, blurred or hazy such that substantially all objects viewed through the obscuration area are undiscernible by a wearer.

In some or all examples, the lenses are subjected to abrasive blasting until an exposed area of the lenses has a haze of 25% or more, in some examples 35% or more, and more in some examples 50% or more.

In some examples, the abrasive blasting is applied to an inner surface of the lenses.

In accordance with further aspects of the present application, there is provided stickers for providing a light blocking, occluding, obscuring or tinted area in the lenses of therapeutic eyeglasses as described herein, and a method of making such stickers as described herein.

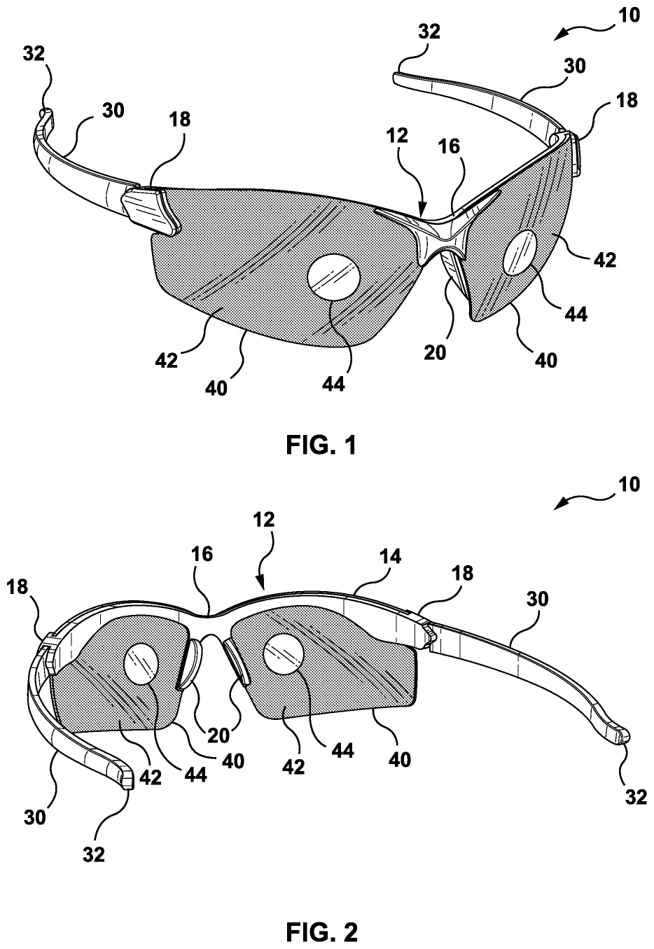

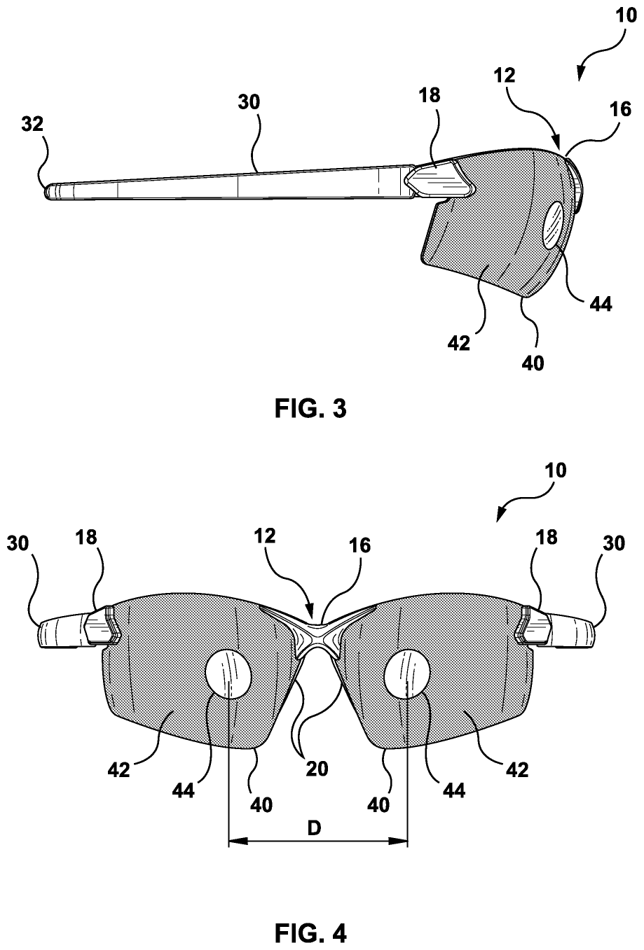

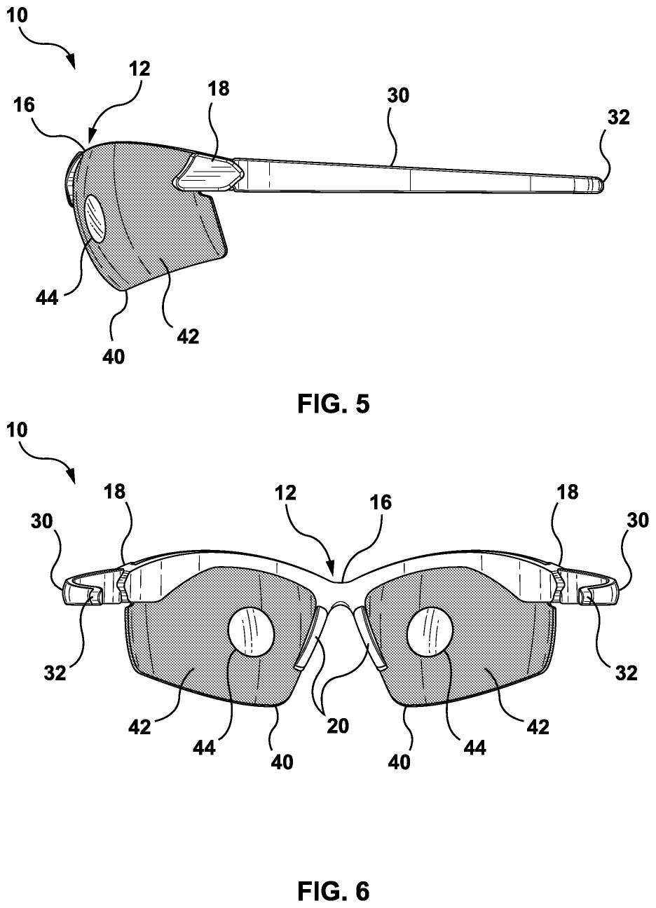

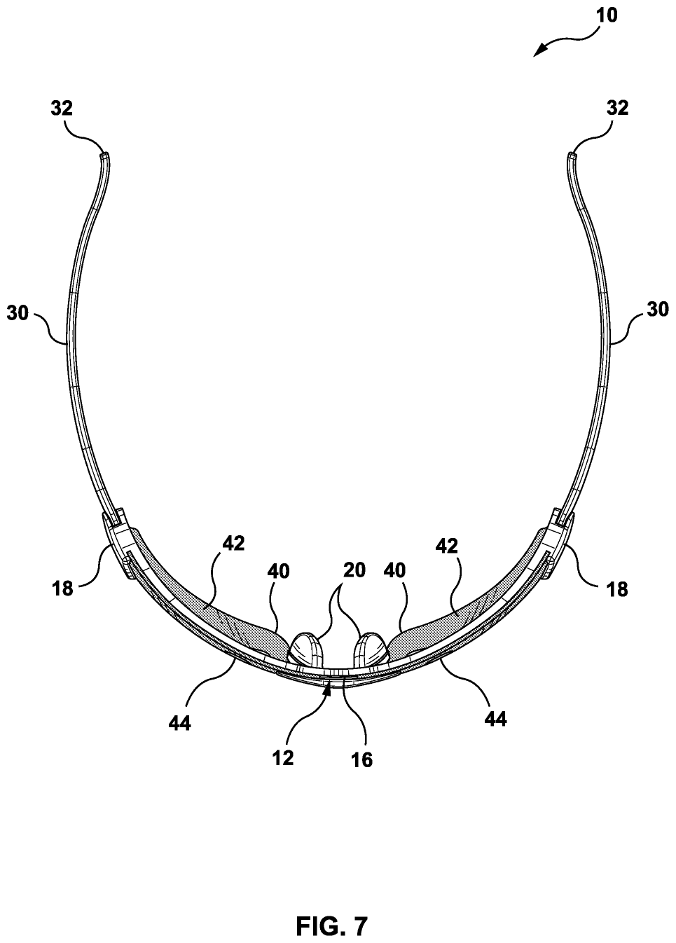

FIGS. 1-7 illustrate therapeutic eyeglasses 10 in accordance with a first embodiment of the present disclosure. The therapeutic eyeglasses 10 comprise a frame 12 comprising a frame front 14, bridge 16, end pieces 18, and nose pads 20. The frame 12 further comprises a pair of temples (also known as arms) 30 attached to the rear side of the frame front at respective ends of the frame front 14 at the end pieces 18 via hinges 22. The temples 30 terminate in a temple tip 32. The temple tips 32 may be used to secure the therapeutic eyeglasses 10 about the ears of a wearer or against the head of a wearer. The end pieces 18 may vary in size and shape, depending on the style of temples on your glasses. A pair of lenses 40 are received in the frame front 14. The lenses 40 may be prescription lenses adapted to modify the vision of a wearer or non-prescription lenses. The lenses 40 may have one or a number of functional coatings, such as Ultraviolet-blocking (UV-blocking), polarizing or scratch-resistant coatings. The lenses 40 may be transparent, coloured, or tinted.

The frame 12 may be shaped and configured similar to the frames of prescription eyeglasses, reading glasses, sunglasses or safety glasses. The frame front 14 provides a style and aesthetic of the therapeutic eyeglasses 10. Although a particular style and aesthetic is shown, the style and aesthetic may vary along with material, colour shape and size. The frame front 14 may be made from various types of material, commonly a rigid plastic such as cellulose acetate, metal or high-performance composite such as carbon fibre. The frame front 14 in the shown embodiment is a half-rim without a lower half such that bottom edges of the lenses 40 are exposed and typically secured in place using a thin nylon chord. However, it is contemplated that a full-rim frame front that covers the entire edge of the lenses or a rimless frame front may be used in other embodiments. In a full-rim face front the lenses 40 are held in place using an angled recess in the frame front called a lens groove. Rimless frame fronts are typically joined together via a metal bridge, with screws in the metal bridge joining and securing the lenses 40 together to form the frame front 14. At the edges of the lenses 40, the temples 30 are also attached via screws through the outer edges of each lens 40.

Each lens 40 comprises a light blocking area 42 and a round, and in particular, circular light transmitting area 44 within the light blocking area 42. The circular light transmitting area 44 may be defined by a two-dimensional (2D) to three-dimensional (3D) projection (2D-to-3D projection) of a circle onto a front surface (outer surface) of the lens 140. The light blocking area 42 blocks the transmission of all, or substantially all, light from being transmitted through the lens 40. In contrast, the circular light transmitting area 44 transmits all, or substantially all, light received at the lens 40. Substantially all light is considered to be 75% or more, in some examples 85-95% of more, of the light which reaches the lens 40.

The circular light transmitting area 44 is proximate to, or near, a centre of the respective lens 30. The circular light transmitting area 44 may be located proximate to an optical centre of each lens 40 and/or a geometric centre of the lens 40. The optical centre is based on a pupillary distance of the wearer while geometric centre is based on the frame 12. In at least some examples, the circular light transmitting area 44 is located below the centre of the respective lens 40, i.e. below the optical centre or a geometric centre of the lens. The circular light transmitting area 44 is located 2 mm to 15 mm below the centre of the respective lens 40 in some examples, 2 mm to 10 mm in some examples, 2 mm to 7.5 mm in some examples, and 2 mm to 5 mm in some examples.

The diameter of the circular light transmitting area 44 is between 10 and 25 mm, in some examples between 15 and 20 mm, and some examples 15 mm, and in some examples 20 mm. The linear distance between the centres of the circular light transmitting area 44 in the lenses 40, denoted D, is between 40 mm and 80 mm, in some examples between 55 mm and 65 mm, and some examples between 57.5 mm and 62.5 mm. In one example, the diameter of the circular light transmitting area 44 is 16 mm, and the distance between the centres of the circular light transmitting area 44 is 58 mm. In another example, the diameter of the circular light transmitting area 44 is 20 mm, and the distance between the centres of the circular light transmitting area 44 is 61 mm.

The light blocking area 42 of each lens may be formed by a durable coating on the lens applied by techniques known in the art. The coating material and thickness may vary. The coating may be made from any suitable coating material that blocks the transmission of all, or substantially all, light from being transmitted through the lens 40. The coating is durable in that it is wear-resistant, water insoluble and water-resistant. Examples of suitable coating material include epoxy-based coatings. Alternatively, rather than a durable coating, the light blocking area 42 and circular light transmitting area 44 may be integrally formed as part of the lens 40 during manufacture.

Alternatively, rather than a durable coating, the light blocking area 42 may be formed by a light blocking film adhered on the lens 40. The light blocking film may be an adhesive-back film that is applied to the lens 40, such as a vinyl sticker providing the light blocking film, or the light blocking film may be attached to the lens 40 using a separate adhesive. The sticker may be custom-made for any set of eyeglasses. In one example, the shape (e.g., size and curvature) of the inside of the lenses 40 is measured, for example, using three-dimensional scanning or other tools, and a sticker may be custom cut (e.g., laser cut) for the inside of the lenses 40 of the eyeglasses, the sticker matching the shape of the inside of the lenses 40 with a circular hole in the sticker providing the circular light transmitting area 44. Thus, the inside shape of the eyeglasses is reverse-engineered, and a custom sticker laser cut to fit the eyeglasses with a hole or window therein for vision of the user. The sticker may then be applied to the inside of the lenses 40 of the eyeglasses. The use of inside or interior surface of the lenses 40 of the eyeglasses rather than the outside reduces the wear-and-tear and exposure of the light blocking film, thereby increasing its useful life. The use of inside or interior surface of the lenses 40 of the eyeglasses also reduces interference with any optical coating (e.g., polarizing, UV blocking, etc.) on the outside or exterior surface of the lens 40. The outside or external surface may be used in other examples if desired.

The circular light transmitting area (or window) 44 may be provided by a clear or transparent area free from any coating, film, abrading or other light modifying treatment, i.e. an uncoated area, untreated area, unabraded of the lens 40 without any light modifying film.

The frame 12 and lenses 40 of the therapeutic eyeglasses 10 are curved in the shown embodiment. In particular, the frame 12 is a wraparound frame having a high degree of curvature and the lenses 40 have a corresponding degree of curvature. The wraparound frame curves around the head of the wearer, reducing the space between the therapeutic eyeglasses 10 and the head of the wearer, thereby limiting peripheral light such as light originating from the top, bottom or sides from reaching the eyes of the wearer. The wraparound frame and the light blocking area 42 cooperate to narrow the field of vision (also known as field of view) and blocking peripheral light and peripheral vision, in contrast the conventional use of wraparound frames to increase the field of vision.

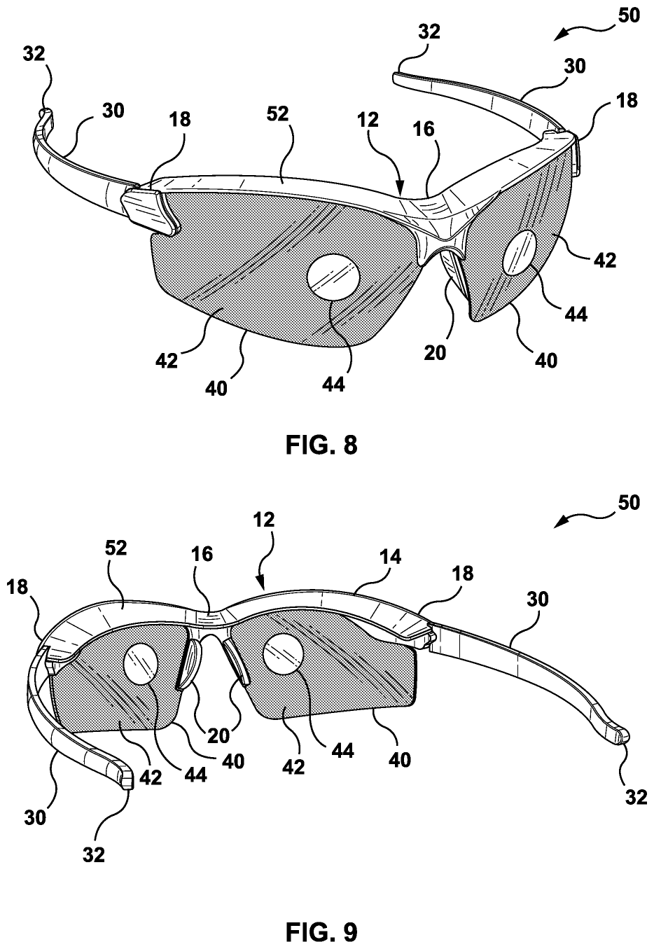

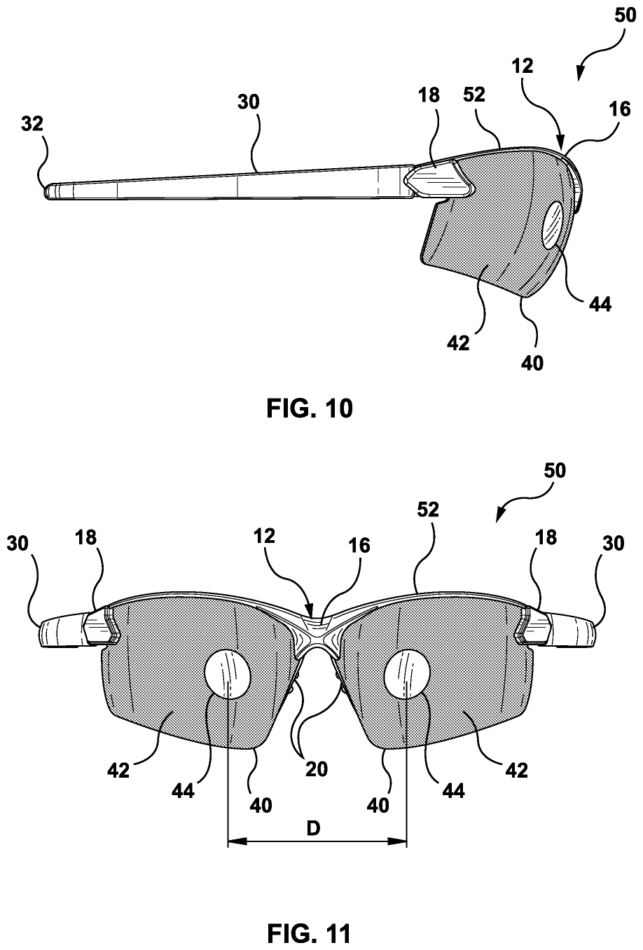

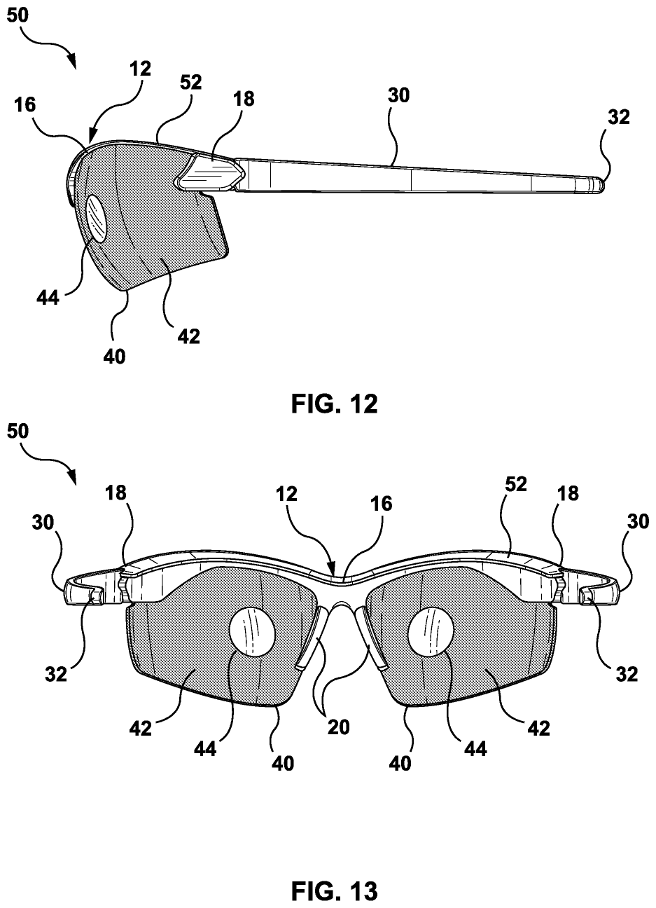

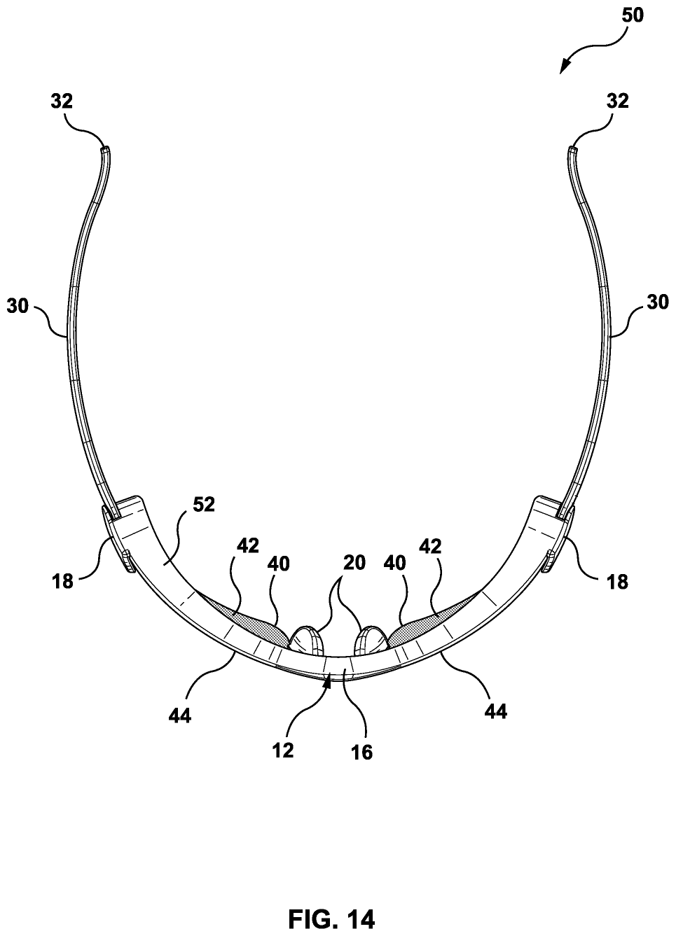

FIGS. 8-14 illustrate therapeutic eyeglasses 50 in accordance with a second embodiment of the present disclosure. The therapeutic eyeglasses 50 are similar to the therapeutic eyeglasses 10. However, the therapeutic eyeglasses 50 further comprise a light blocking top shield 52 extending from a top of the frame front 14 to block peripheral light entering from the top of the frame 12 from the eyes of a wearer. The light blocking top shield 52 may be integrally formed as part of the front frame 14.

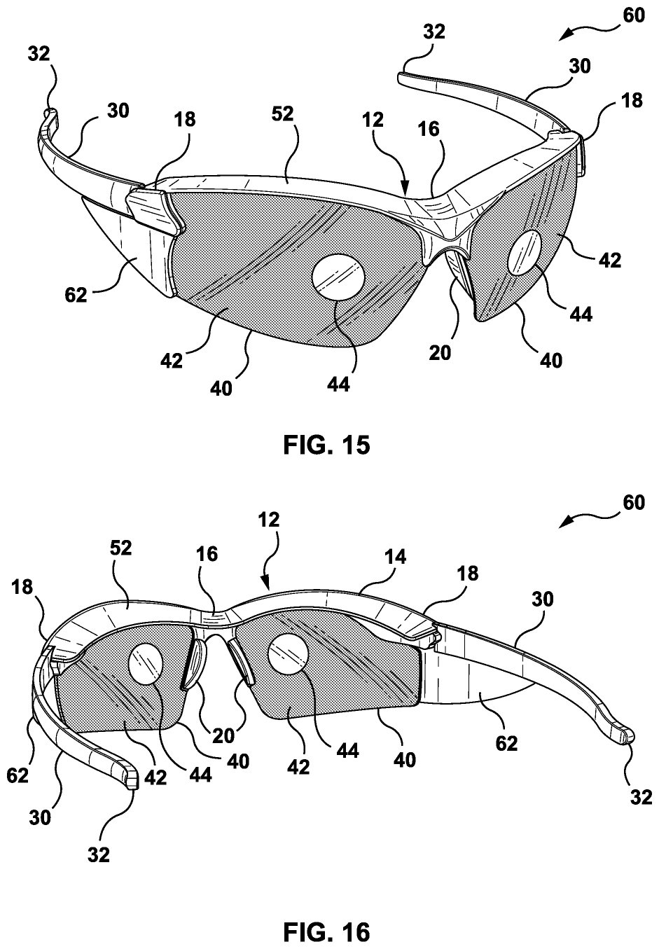

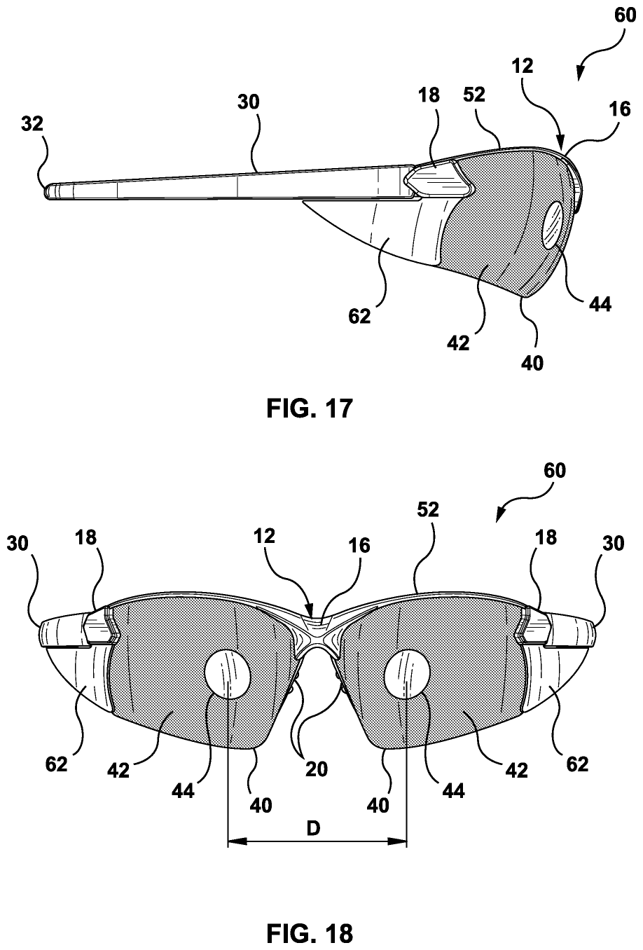

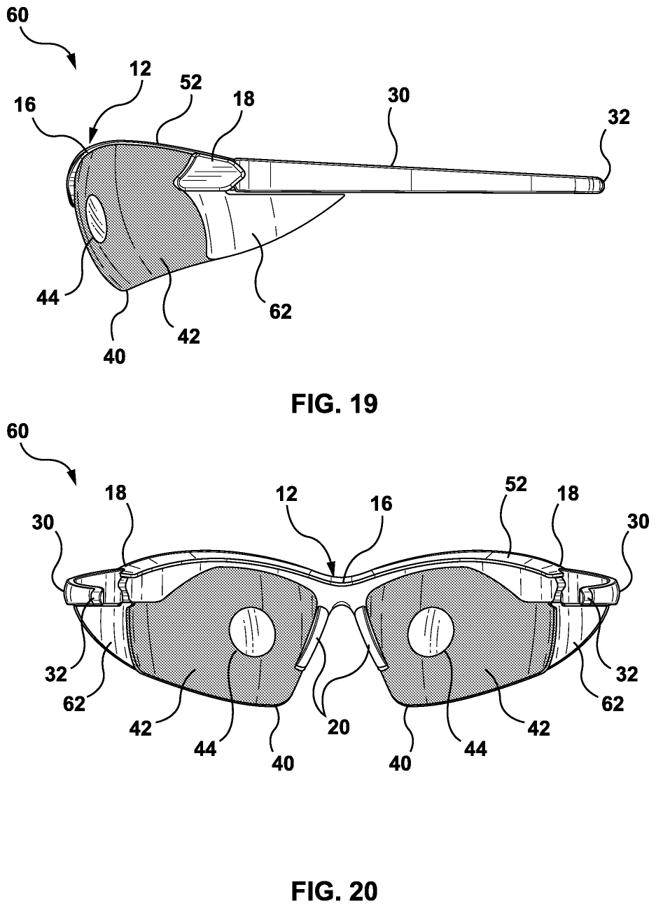

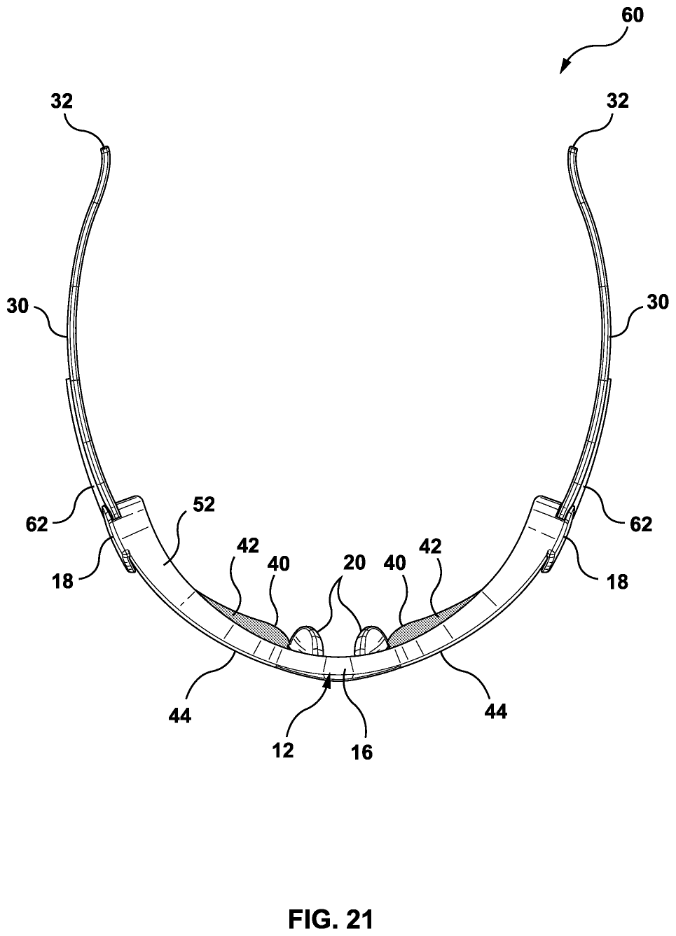

FIGS. 15-21 illustrate therapeutic eyeglasses 60 in accordance with a third embodiment of the present disclosure. The therapeutic eyeglasses 60 are similar to the therapeutic eyeglasses 50. However, the therapeutic eyeglasses 60 further comprise light blocking side shields 62 extending from each of the temples 30 to block peripheral light entering from the sides of the frame 12 from the eyes of a wearer. The light blocking side shields 62 may be two-part shields comprised of an upper and lower side shield. The light blocking side shields 62 may be integrally formed as part of the temples 30.

In one alternative embodiment, rather than each lens 40 comprising a light blocking area 42 and a circular light transmitting area 44 within the light blocking area 42, each lens 40 may comprise a tinted area and a circular non-tinted area within the tinted area, a highly tinted area and a circular lowly tinted area within the highly tinted area, or coloured area and a circular non-coloured area within the highly tinted area having the same or similar dimensions to the light blocking area 42 and circular light transmitting area 44 described above.

In another alternative embodiments, each lens may comprise an obscuration area (also known as a light diffusing area) and a circular light transmitting area within the obscuration area. The obscuration area scatters and/or diffuses light received by the lens such that the vision of a wearer is substantially obscured, blurred or hazy, making it difficult for the wearer to discern anything viewed through the obscuration area. The obscuration area of each lens is provided by an abraded surface. The abraded surface may be formed by abrasive process, such as abrasive blasting, on a surface of the lens. The abrasive process is typically performed on the inside or interior surface of the lens so as to preserve any optical coating (e.g., polarizing, UV blocking, etc.) on the outside or exterior surface of the lens. However, the abrasive process may be performed on the outside or exterior surface of the lens if desired. Abrasive blasting is the process of forcing solid particles across a surface at high speeds to abrade the surface, such as sandblasting. In one example, a mask or similar covering having the same size and shape of the light transmitting area of the therapeutic eyeglasses is temporally placed over the lenses. The mask may be provided by a vinyl sticker. The lenses are then subjected to abrasive blasting until a threshold level of abrasion is achieved. The threshold level of abrasion is achieved when a threshold level of obscuration is achieved. The threshold level of obscuration is achieved when light passing through the obscuration area is sufficiently scattered, blurred or hazy such that substantially all objects viewed through the obscuration area are undiscernible by the wearer. Obstruction is related to a haze of the lens in the obscuration area. Haze is typically measured as a percentage of light scattered, and ranges between 0% and 100% in accordance with standards ASTM D1003/D1044 and ISO13468/ISO14782, which are incorporated herein by reference. In some examples, the obscuration area has a haze of 25% or more, in some examples 35% or more, and more in some examples 50% or more.

The abraded surface may be formed on the lens before or after insertion into the eyeglasses. For example, the abraded lens may be made by a lens manufacturer to be provided to eyeglass makers or opticians, by eyeglass makers or opticians during assemble or manufacture of the eyeglasses, or by opticians or other service providers who can perform the abrasive processing on any eyeglasses provided by the wearer.

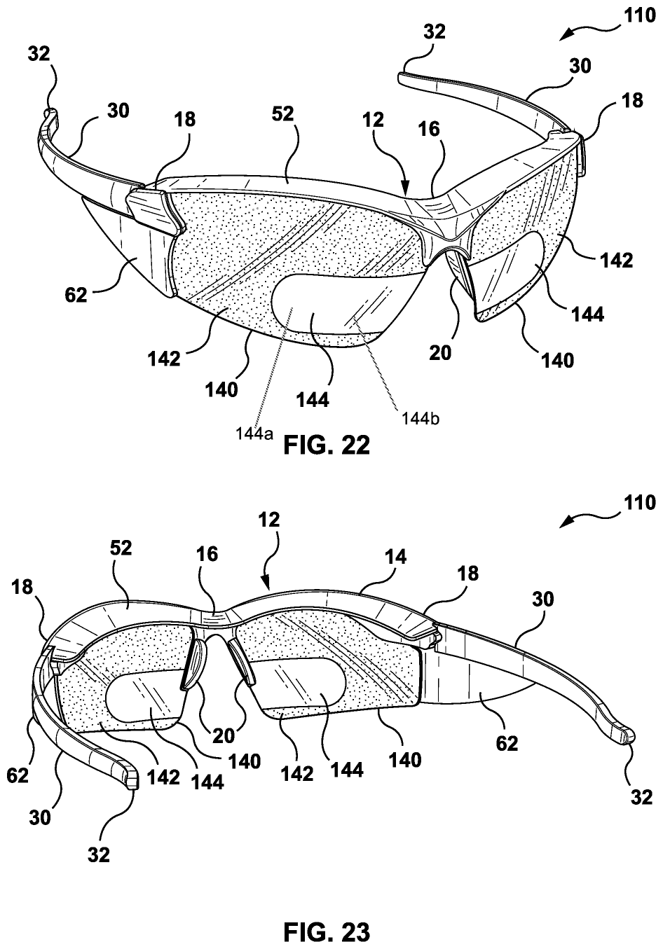

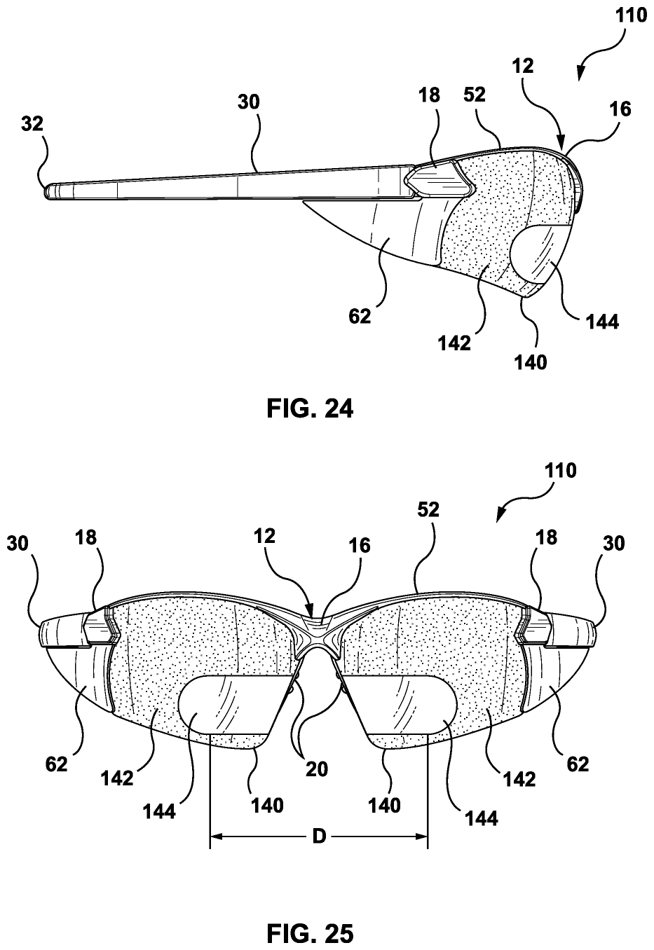

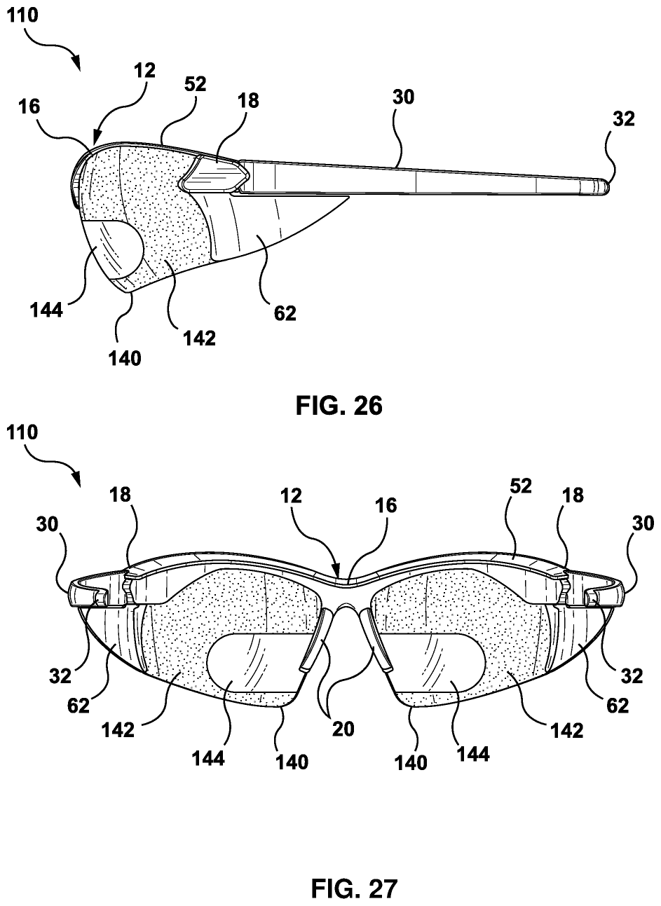

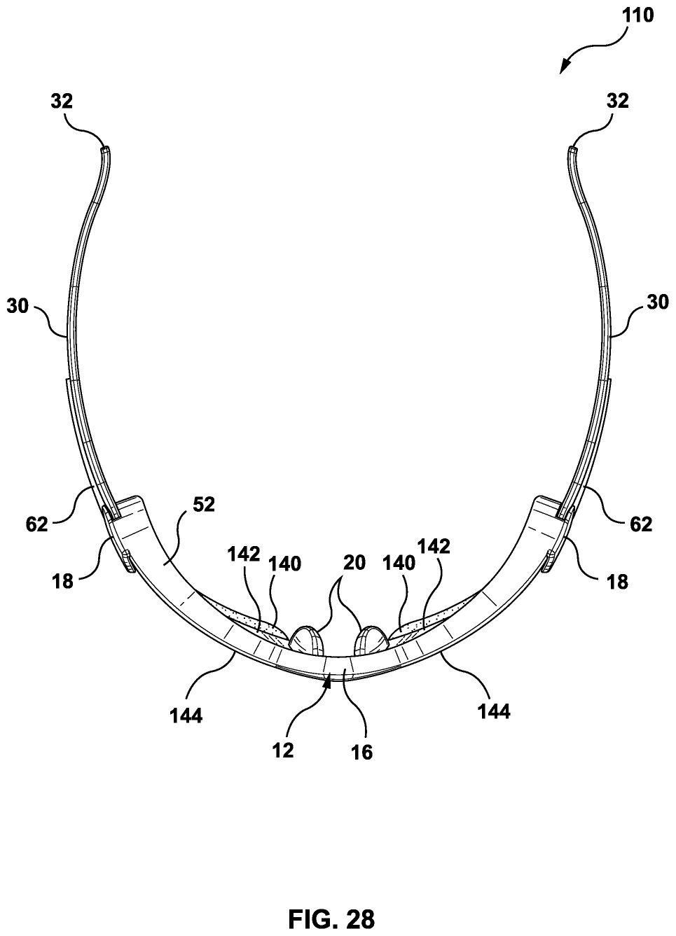

FIGS. 22-28 illustrate therapeutic eyeglasses 110 in accordance with a fourth embodiment of the present disclosure. The therapeutic eyeglasses 110 are similar to the therapeutic eyeglasses 60 except that an obscuration area 142 is provided in each lens 140 rather than a light blocking area. An elongate light transmitting area (or slotted light transmitting area) 144 is provided within each obscuration area 142. The obscuration area of the therapeutic eyeglasses 110 is similar to that described above. The elongate light transmitting area 144 comprises a curved portion (or round portion) 144a having a centre and an elongate portion 144b extending laterally from the curved portion 144a to an inner edge of the lens 140 proximate to the bridge 16. In the shown example, the light transmitting area 144 is defined by a 2D-to-3D projection of a semi-circle adjoined to a generally trapezoidal shape elongate portion onto a front surface (outer surface) of the lens 140. The centre of the curved portion 144a of the elongate light transmitting area 144 may be proximate to a centre of each lens 140. The centre of the curved portion 144a of the elongate light transmitting area 144 may be located proximate to an optical centre of each lens 140 and/or a geometric centre of each lens 140. In at least some examples, the centre of the curved portion 144a of the elongate light transmitting area 144 is located below the centre of the respective lens 140, i.e. below the optical centre or a geometric centre of the lens. The elongate light transmitting area 144 is located 2 mm to 15 mm below the centre of the respective lens 40 in some examples, 2 mm to 10 mm in some examples, 2 mm to 7.5 mm in some examples, and 2 mm to 5 mm in some examples. The distance between the upper and lower lines of the elongate portion 144b is the same as the diameter of the semi-circular curved portion 144a.

In the therapeutic eyeglasses 110, the elongate light transmitting areas 144 extend laterally across the lens 140 so that when the therapeutic eyeglasses 110 are worn by a user the elongate light transmitting area 144 extends horizontally across the therapeutic eyeglasses 110, the elongate portion 144b forming a generally trapezoidal shape elongate portion defined by a pair of parallel straight lines extending laterally from the curved portion 166a to an inner edge of the lens 140 proximate to the bridge 16. It will be appreciated that the elongate light transmitting area 144 may be extend in a direction a few degrees from the horizontal when the therapeutic eyeglasses 110 are worn by a user and still be considered to extend horizontally.

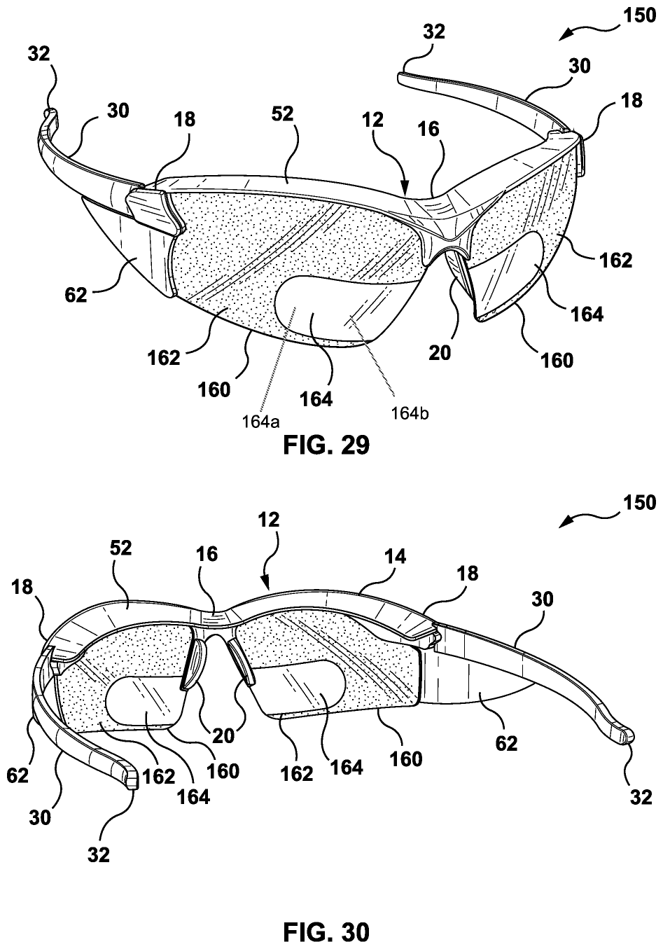

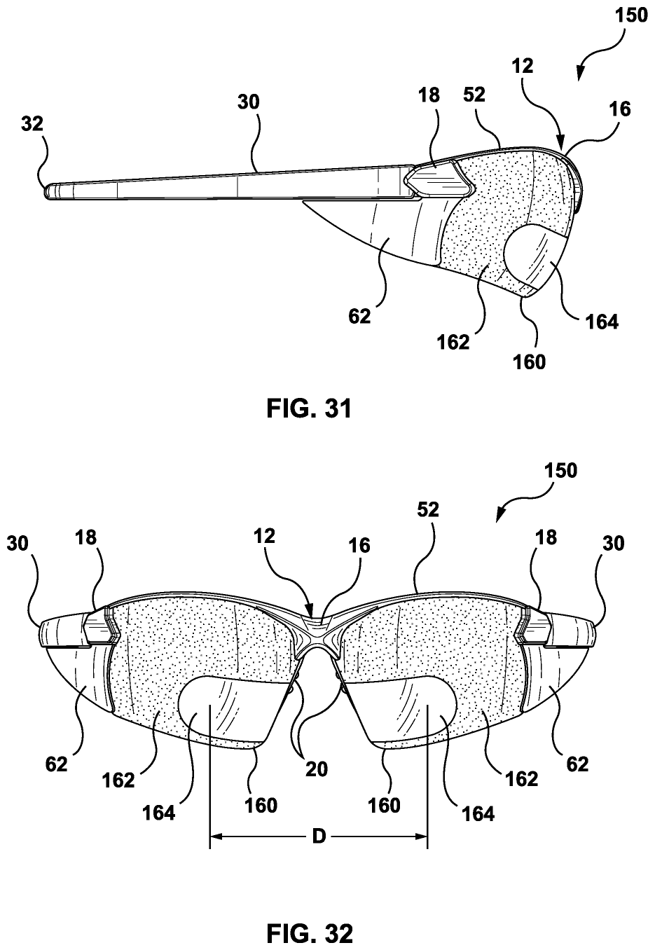

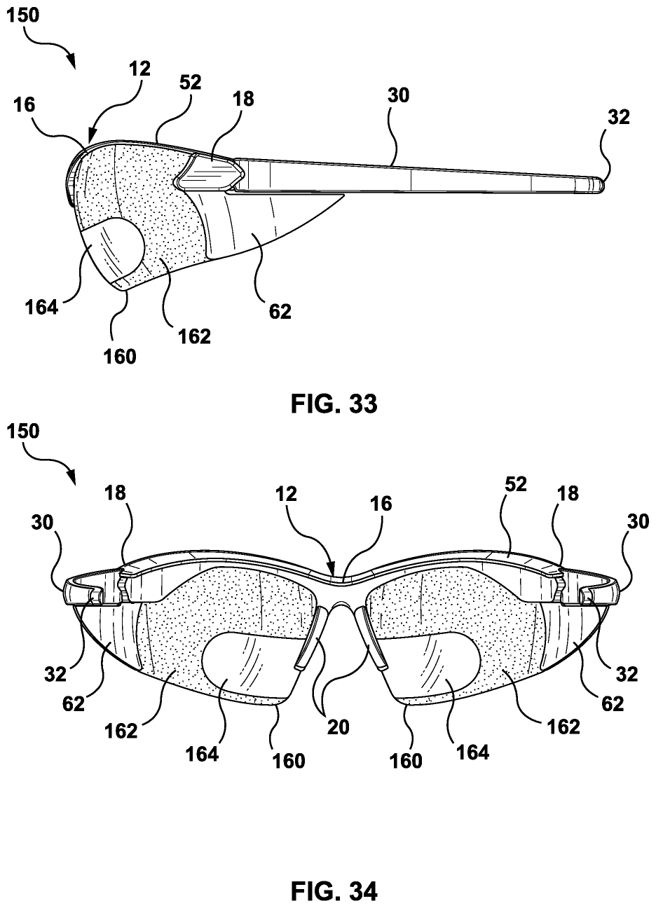

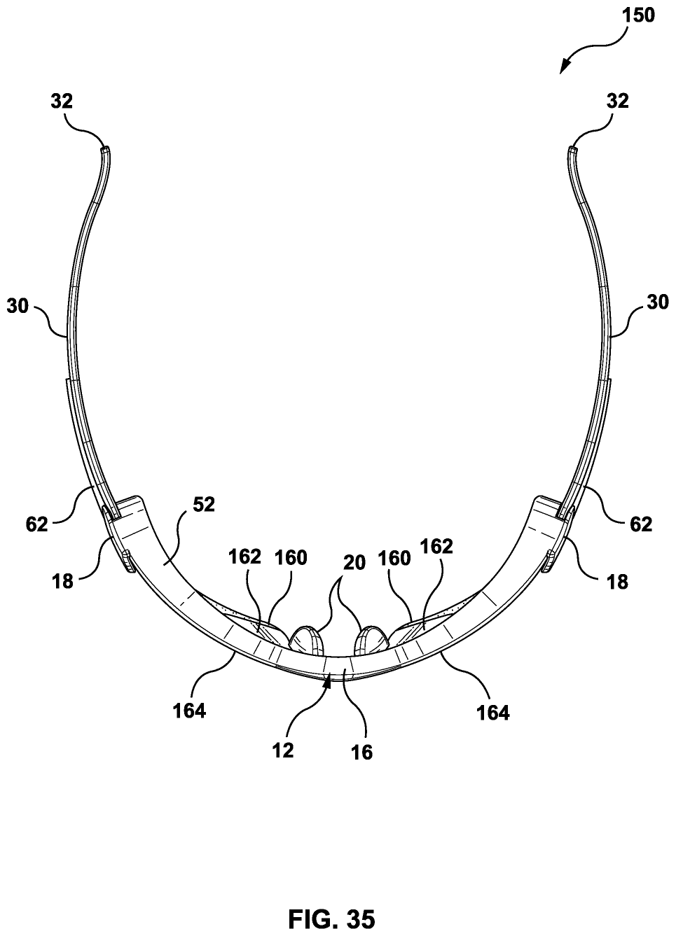

FIGS. 29-35 illustrate therapeutic eyeglasses 150 in accordance with a fifth embodiment of the present disclosure. The therapeutic eyeglasses 150 are similar to the therapeutic eyeglasses 150 except that the shape of the obscuration area differs. In the therapeutic eyeglasses 150, there is provided an obscuration area 162 in each lens 160 with an elongate light transmitting area (or slotted light transmitting area) 164 provided within each obscuration area 162. The elongate light transmitting area 164 comprises a curved portion (or round portion) 164a having a centre and an elongate portion 164b extending laterally from the curved portion 166a to an inner edge of the lens 140 proximate to the bridge 16. The centre of the curved portion 164a of the elongate light transmitting area 164 may be proximate to a centre of each lens 160. The centre of the curved portion 164a of the elongate light transmitting area 164 may be located proximate to an optical centre of each lens 160 and/or a geometric centre of each lens 160. In at least some examples, the centre of the curved portion 164a of the elongate light transmitting area 164 is located below the centre of the respective lens 160, i.e. below the optical centre or a geometric centre of the lens. The elongate light transmitting area 164 is located 2 mm to 15 mm below the centre of the respective lens 40 in some examples, 2 mm to 10 mm in some examples, 2 mm to 7.5 mm in some examples, and 2 mm to 5 mm in some examples.

In the therapeutic eyeglasses 150, the elongate light transmitting areas 164 extend laterally across the lens 160 so that when the therapeutic eyeglasses 150 are worn by a user the elongate light transmitting area 164 extends generally horizontally across the therapeutic eyeglasses 110 the elongate portion 144b forming a generally trapezoidal shape elongate portion defined by a pair of parallel curved lines (parallel curves) extending laterally from the curved portion 166a to an inner edge of the lens 140 proximate to the bridge 16. It will be appreciated that the elongate light transmitting area 164 may be extend in a direction a few degrees from the horizontal when the therapeutic eyeglasses 150 are worn by a user and still be considered to extend generally horizontally.

The linear distance between the centre of the curved portions 144a, 164a of the elongate light transmitting areas 144, 162 in each lens 140, 160, is denoted D, and may vary between 40 mm and 80 mm, in some examples between 55 mm and 65 mm, and some examples between 57.5 mm and 62.5 mm. The diameter of the semi-circular portion is between 10 and 25 mm, and in some examples, between 15 and 20 mm, and some examples 15 mm, and in some examples 20 mm. In one example, the linear distance between the centre of the curved portions 144a, 164a of the elongate light transmitting areas 144, 162 in each lens 140, 160, is 61 mm and the diameter of the semi-circular portion is 20 mm. In another one example, the linear distance between the centre of the curved portions 144a, 164a of the elongate light transmitting areas 144, 162 in each lens 140, 160, is 61 mm and the diameter of the semi-circular portion is 15 mm.

The elongate light transmitting areas 144, 162 may be provided by a clear or transparent area free from any coating, film, abrading or other light modifying treatment, i.e. an uncoated area, untreated area, unabraded of the lens 140, 160 without any light modifying film.

The configuration of the light transmitting area 44, 144 and 164 of the therapeutic eyeglasses is independent of the frame 12 or the respective lens. The configuration of light transmitting area 44, 144 and 164, for example size and shape in the above-described embodiments, may be adapted for use by most wearers irrespective of differences in the eye size and/or pupillary distance, which may vary based on the size, age and face shape of the wearer, among other possible factors. Alternatively, in other embodiments the configuration of light transmitting area 44, 144 and 164, for example size and shape, may be customized for the eye size and/or pupillary distance of target wearer.

The therapeutic eyeglasses of the present disclosure reduce peripheral vision, thereby allowing the wearer to focus better because peripheral visual distractions are limited. The therapeutic eyeglasses of the present disclosure provide one or more therapeutic benefits to wearers. It will be understood to persons skilled in the art that scope and nature of therapeutic benefits are difficult to predict or unpredictable. Based on testing performed by the applicant, the benefits of reduced peripheral vision provided by the therapeutic eyeglasses of the present disclosure include increased focus and concentration, which can lead to increased efficiency and productivity in relation to mental tasks such as school or office work. The increased focus and concentration may also be useful in sporting applications such as a sports training device in golf, baseball, hockey, or the like. In one study of 10 golfers, use of the therapeutic eyeglasses with circular light transmitting areas such as in FIG. 1-21 described above showed an average of 18% increase in putts made 4 ft. from the hole. Other benefits may include reduced stress, anxiety, and panic attacks and a feeling of relaxation in some users, such as persons with ADHD. The therapeutic eyeglasses may also reduce eye strain in computer users and reduce discomfort for users with light sensitivity. The benefits of the therapeutic eyeglasses may be enduring beyond the time that the therapeutic eyeglasses are worn. In one study with 10 students aged 14 to 16 diagnosed ADHD, use of the therapeutic eyeglasses with circular light transmitting areas such as in FIG. 1-21 described above significantly reduced distraction and increased focus while the students were working at their desk, the hallway, and at a computer. The students were observed by the teacher having longer states of concentration on individual tasks.

The therapeutic eyeglasses of the fourth and fifth embodiments may be more advantageous for applications in which the wearer has a shorter (or shallower) depth of field such as when the wearer is working at desk (e.g., doing paperwork/schoolwork, reading, or working on a computer), reading, or working with their hands as a result of the inner light transmitting area (e.g., non-light blocking, non-occluding, non-obscuring or non-tinted area) provided by the elongate portion of the elongate light transmitting area compared with the therapeutic eyeglasses of the earlier embodiments which lack the inner light transmitting area and may be more advantageous for applications in which the wearer has a longer (or deeper) depth of field such as when the wearer is playing sports.

All values and sub-ranges within disclosed ranges are also disclosed. Features from one or more of the above-described embodiments may be selected to create alternate embodiments comprised of a subcombination of features which may not be explicitly described above. In addition, features from one or more of the above-described embodiments may be selected and combined to create alternate embodiments comprised of a combination of features which may not be explicitly described above. Features suitable for such combinations and subcombinations would be readily apparent to persons skilled in the art upon review of the present disclosure as a whole.

The present disclosure may be embodied in other specific forms without departing from the subject matter of the claims. The described example embodiments are to be considered in all respects as being only illustrative and not restrictive. The present disclosure intends to cover and embrace all suitable changes in technology. The scope of the present disclosure is, therefore, described by the appended claims rather than by the foregoing description. The scope of the claims should not be limited by the preferred embodiments set forth in the examples, but should be given the broadest interpretation consistent with the description as a whole.

1. Therapeutic eyeglasses, comprising:
a frame comprising:
a frame front; and
a pair of temples attached to respective ends of the frame front; and
a pair of lenses received in the frame front, wherein each lens comprises a light modifying area and a circular light transmitting area within the light modifying area, the circular light transmitting area proximate to a centre of the lens, wherein the diameter of the circular light transmitting area is between 10 and 25 mm, and a linear distance between a centre of the circular light transmitting area of the lenses is between 40 mm and 80 mm.

2. The therapeutic eyeglasses of claim 1, wherein the diameter of the circular light transmitting area is between 15 and 20 mm.

3. The therapeutic eyeglasses of claim 1, wherein the linear distance between the centre of the circular light transmitting area is between 55 mm and 65 mm.

4. The therapeutic eyeglasses of claim 1, wherein the linear distance between the centre of the circular light transmitting area is between 57.5 mm and 62.5 mm.

5. The therapeutic eyeglasses of claim 1, wherein the circular light transmitting area is located below the centre of the respective lens.

6. Therapeutic eyeglasses, comprising:
a frame comprising:
a frame front comprising a bridge; and
a pair of temples attached to respective ends of the frame front; and
a pair of lenses received in the frame front, wherein each lens comprises a light modifying area and an elongate light transmitting area within the light modifying area, the elongate light transmitting area comprising a curved portion having a centre and an elongate portion extending laterally from the curved portion to an inner edge of the lens proximate to the bridge, the centre of the curved portion of the elongate light transmitting area proximate to a centre of each lens, wherein a linear distance between the centre of the curved portion of the elongate light transmitting area of the lenses is between 40 mm and 80 mm.

7. The therapeutic eyeglasses of claim 6, wherein the curved portion is a semi-circular portion.

8. The therapeutic eyeglasses of claim 7, wherein the diameter of the semi-circular portion is between 10 and 25 mm.

9. The therapeutic eyeglasses of claim 8, wherein the diameter of the semi-circular portion is between 15 and 20 mm.

10. The therapeutic eyeglasses of claim 6, wherein the linear distance between the centre of the curved portions of the elongate light transmitting areas in each lens is between 55 mm and 65 mm.

11. The therapeutic eyeglasses of claim 6, wherein the linear distance between the centre of the curved portions of the elongate light transmitting areas in each lens is between 57.5 mm and 62.5 mm.

12. The therapeutic eyeglasses of claim 6, wherein the elongate light transmitting area is located below the centre of the respective lens.

13. The therapeutic eyeglasses of claim 1, wherein the frame and lenses are curved.

14. The therapeutic eyeglasses of claim 13, wherein the therapeutic eyeglasses are wraparound eyeglasses.

15. The therapeutic eyeglasses of claim 1, wherein the frame further comprises:
a light blocking top shield extending from a top of the frame front to block peripheral light from the eyes of a wearer; and/or
light blocking side shields extending from each of the temples to block peripheral light from the eyes of a wearer.

16. The therapeutic eyeglasses of claim 1, wherein the light modifying area of each lens is formed by a durable coating on the lens or a film adhered on the lens.

17. The therapeutic eyeglasses of claim 1, wherein the light modifying area is:
a light blocking area that prevents substantially all light from passing through the lens;
a tinted area and the circular light transmitting area is a circular non-tinted area within the tinted area; or
an obscuration area that obscures the vision of a wearer, wherein in some examples the obscuration area of each lens is provided by an abraded surface.

18. The therapeutic eyeglasses of claim 17, wherein the obscuration area scatters and/or diffuses light such that substantially all objects viewed through the obscuration area are undiscernible by the wearer.

19. The therapeutic eyeglasses of claim 17, wherein the obscuration area has a haze of 25% or more, in some examples 35% or more, and more in some examples 50% or more.

20. A method of making therapeutic eyeglasses, comprising:
inserting a pair of lenses into a frame of the therapeutic eyeglasses;
applying a mask to a surface of each lens in the pair of lenses of the therapeutic eyeglasses, wherein each mask has a size and shape corresponding to a size and shape of a light transmitting area of a respective lens;
abrading the surface of each lens; and
removing the mask from each lens in the pair of lenses.
